(19) United States
(12) Patent Application Publication
(10) Pub. No.: US 2025/0191769 A1
(43) Pub. Date: Jun. 12, 2025

(54) SYSTEMS AND METHODS FOR SIMULATION AND ASSESSMENT OF DAILY ACTIVITIES IN A VIRTUAL ENVIRONMENT

(71) Applicant: Neuromersive, Inc., Fort Worth, TX (US)

(72) Inventors: Veena Somareddy, Euless, TX (US); Salome Jesus-Jose Marquez, Eulesss, TX (US)

(73) Assignee: Neuromersive, Inc., Fort Worth, TX (US)

(21) Appl. No.: 18/533,802

(22) Filed: Dec. 8, 2023

(51) Int. Cl. G16H 50/30; G16H 40/67

(52) U.S. Cl. CPC G16H 50/30; G16H 40/67

(57) ABSTRACT

Systems and methods for simulating and assessing performance of real-world activities within a virtual environment by a therapy patient. In embodiments, an activity of daily living (ADL) is represented a plurality of virtual tasks. Each of the virtual tasks may be associated with the ADL such that a performance of the virtual tasks is indicative of a likelihood that the ADL can be performed safely and effectively by the therapy patient. A virtual environment configured to enable performance of the virtual tasks is generated, and data indicative of performance of each virtual task by the therapy patient is received. In embodiments, whether the therapy patient is able to perform the real-world ADL safely and effectively may be determined based on the data indicative of the performance of each virtual task by the therapy patient.

### TECHNICAL FIELD

The present disclosure relates generally to therapy technologies, and more particularly to systems and methods for simulating and assessing performance of real-world activities within a virtual environment by a therapy patient with brain injury.

### BACKGROUND

Many people suffer from traumatic injuries that affect their ability to perform everyday activities. These injuries, resulting from various causes such as accidents, falls, or sports-related events, can lead to long-term disabilities, significantly altering the course of a person's life. Among these, traumatic brain injuries are particularly impactful. The severity of a brain injury can range from a mild concussion to a severe injury, affecting cognitive functions, physical abilities, emotional stability, and social interactions. Individuals with brain injuries may find themselves struggling with tasks that were once effortless. Simple tasks such as going to the toilet, dressing, personal grooming, cooking, and managing household chores become daunting, greatly impacting the patient's independence and emotional well-being. This disruption in basic daily functioning affects not only the individuals but also their families, who often struggle to adapt to these changes.

Luckily for many, effective therapy and rehabilitation systems have been developed that allow many of those with brain injuries (and other traumatic injuries) to recover from their injuries and to be again able to perform activities of daily living (ADLs). However, the road to recovery can be long, arduous, and expensive, and it is desirable to minimize the time a therapy patient has to spend in a therapy program. Deciding when a therapy patient can be released from a therapy program, or even deciding to allow a patient to perform certain activities that may be currently contraindicated to the patient, can be a difficult process that comes with a unique set of challenges.

Assessing a patient's readiness to resume daily activities unsupervised is a critical component of the rehabilitation process, yet it is fraught with complexities. Hospitals, for example, are not designed to accurately simulate a home environment, making in-hospital assessments less effective. The alternative of transporting patients to external environments for more realistic assessments presents logistical and resource challenges. Dedicated testing environments, while potentially more realistic, often require significant space and resources and may still not reflect a patient's actual home setting accurately. Home-based assessments, though ideal in theory, involve logistical challenges such as the risks associated with premature patient discharge, and the requirement for therapists to travel to the patient's home.

Furthermore, safety is a paramount concern during these assessments, particularly for tasks that could pose risks. Activities that involve cooking or handling potentially dangerous tools can be hazardous for individuals with impaired motor skills or cognitive functions, highlighting the need for safe and effective assessment methods. Moreover, assessing a patient's response to a dangerous situation (e.g., a stove left on, smoke, a gas leak, etc.) without exposing the patient to the dangerous situation may not be possible, and may limit the value of the assessment.

The current state of patient assessment in this context underscores the need for innovative approaches. Currently, there are simply no therapy systems that enable simulation of real-life environments within a controlled setting, that prioritize patient safety, and that are adaptable to the unique needs and home environments of individual patients, all while minimizing logistical burdens.

### BRIEF SUMMARY

The present disclosure achieves technical advantages as systems and methods that provide functionality for simulating and assessing performance of real-world activities within a virtual environment by a therapy patient. In embodiments, an activity of daily living (ADL), which may represent a real-world activity, may be represented as a plurality of target physical tasks. A plurality of target virtual tasks corresponding to the plurality of target physical tasks may be generated. Each of the plurality of target virtual tasks may be associated with a target physical task of the plurality of target physical tasks such that a performance of a target virtual task of the plurality of target virtual tasks is indicative of a performance of a target physical task. A virtual environment configured to enable performance of the plurality of target virtual tasks may be generated, and data indicative of performance of each target virtual task of the plurality of target virtual tasks by the therapy patient may be received. In embodiments, whether the therapy patient is able to perform the real-world activity successfully may be determined based on the data indicative of the performance of each target virtual task of the plurality of target virtual tasks by the therapy patient with the brain injury. Thus, the present disclosure provides for a system integrated into a practical application with meaningful limitations as an integrated system with functionality for simulating ADLs within a virtual environment that may enable a therapy patient to improve or experience functional independence for performing the ADLs, and may enable a therapist to assess the virtual performance of the ADLs (e.g., in the virtual environment) by the patient in order to determine whether the patient is able to perform the ADLs safely and successfully in the real-world, without exposing the therapy patient to the real-world activity and while being able to perform the simulation and/or assessment in any real-world setting (e.g., a clinical or hospital setting). In this manner, a system implemented in accordance with embodiments herein may provide an improvement to the field of therapy and clinical assessments.

Thus, it will be appreciated that the technological solutions provided herein, and missing from conventional systems, are more than a mere application of a manual process to a computerized environment, but rather include functionality to implement a technical process to replace or supplement current solutions or non-existing solutions for simulating and assessing performance of real-world activities within a virtual environment by a therapy patient. In doing so, the present disclosure goes well beyond a mere application the manual process to a computer. Accordingly, the claims herein necessarily provide a technological solution that overcomes a technological problem.

It is an object of the disclosure to provide a method of simulating and assessing performance of real-world activities within a virtual environment by a therapy patient with brain injury. It is a further object of the disclosure to provide a system for simulating and assessing performance of real-world activities within a virtual environment by a therapy patient with brain injury, and a computer-based tool for simulating and assessing performance of real-world activities within a virtual environment by a physical patient with brain injury. These and other objects are provided by the present disclosure, including at least the following embodiments.

In one particular embodiment, a method of simulating and assessing performance of real-world activities within a virtual environment by a therapy patient with brain injury is provided. The method includes representing a real-world activity as a plurality of target physical tasks, and generating a plurality of target virtual tasks corresponding to the plurality of target physical tasks. In embodiments, each of the plurality of target virtual tasks is associated with a target physical task of the plurality of target physical tasks such that a performance of a target virtual task of the plurality of target virtual tasks is indicative of a performance of a target physical task. The method also includes generating a virtual environment configured to enable performance of the plurality of target virtual tasks, receiving data indicative of performance of each target virtual task of the plurality of target virtual tasks by the therapy patient, and determining, based on the data indicative of the performance of each target virtual task of the plurality of target virtual tasks by the therapy patient, whether the therapy patient is able to perform the real-world activity successfully.

In another embodiment, a system for simulating and assessing performance of real-world activities within a virtual environment by a therapy patient with brain injury is provided. The system comprises at least one processor and a memory operably coupled to the at least one processor and storing processor-readable code that, when executed by the at least one processor, is configured to perform operations. The operations include representing a real-world activity as a plurality of target physical tasks, and generating a plurality of target virtual tasks corresponding to the plurality of target physical tasks. In embodiments, each of the plurality of target virtual tasks is associated with a target physical task of the plurality of target physical tasks such that a performance of a target virtual task of the plurality of target virtual tasks is indicative of a performance of a target physical task. The operations also include generating a virtual environment configured to enable performance of the plurality of target virtual tasks, receiving data indicative of performance of each target virtual task of the plurality of target virtual tasks by the therapy patient, and determining, based on the data indicative of the performance of each target virtual task of the plurality of target virtual tasks by the therapy patient, whether the therapy patient is able to perform the real-world activity successfully.

In yet another embodiment, a computer-based tool for simulating and assessing performance of real-world activities within a virtual environment by a therapy patient with brain injury is provided. The computer-based tool including non-transitory computer readable media having stored thereon computer code which, when executed by a processor, causes a computing device to perform operations. The operations include representing a real-world activity as a plurality of target physical tasks, and generating a plurality of target virtual tasks corresponding to the plurality of target physical tasks. In embodiments, each of the plurality of target virtual tasks is associated with a target physical task of the plurality of target physical tasks such that a performance of a target virtual task of the plurality of target virtual tasks is indicative of a performance of a target physical task. The operations also include generating a virtual environment configured to enable performance of the plurality of target virtual tasks, receiving data indicative of performance of each target virtual task of the plurality of target virtual tasks by the therapy patient, and determining, based on the data indicative of the performance of each target virtual task of the plurality of target virtual tasks by the therapy patient, whether the therapy patient is able to perform the real-world activity successfully.

The foregoing has outlined rather broadly the features and technical advantages of the present disclosure in order that the detailed description that follows may be better understood. Additional features and advantages of the disclosure will be described hereinafter which form the subject of the claims herein. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

FIG. 1 is a block diagram of an exemplary system configured with capabilities and functionality for simulating and assessing performance of real-world activities within a virtual environment by a therapy patient in accordance with embodiments of the present disclosure.

FIG. 2 illustrates an example embodiment implemented in a system in accordance with aspects of the present disclosure for providing functionality for simulating and assessing performance of real-world activities within a virtual environment by a therapy patient in accordance with embodiments of the present disclosure.

FIG. 3A shows an example of a plurality of target virtual tasks generated to represent a real-world activity in accordance with embodiments of the present disclosure.

FIG. 3B illustrates a report including data indicative of performance of virtual tasks of a plurality of virtual tasks associated with a real-world activity in accordance with embodiments of the present disclosure.

FIG. 3C illustrates a report including data related to the overall performance of real-world activities in accordance with embodiments of the present disclosure.

FIG. 4 shows a high-level flow diagram of operation of a system configured to provide functionality for simulating and assessing performance of real-world activities within a virtual environment by a therapy patient in accordance with embodiments of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

The disclosure presented in the following written description and the various features and advantageous details thereof, are explained more fully with reference to the non-limiting examples included in the accompanying drawings and as detailed in the description. Descriptions of well-known components have been omitted to not unnecessarily obscure the principal features de-scribed herein. The examples used in the following description are intended to facilitate an understanding of the ways in which the disclosure can be implemented and practiced. A person of ordinary skill in the art would read this disclosure to mean that any suitable combination of the functionality or exemplary embodiments below could be combined to achieve the subject matter claimed. The disclosure includes either a representative number of species falling within the scope of the genus or structural features common to the members of the genus so that one of ordinary skill in the art can recognize the members of the genus. Accordingly, these examples should not be construed as limiting the scope of the claims.

A person of ordinary skill in the art would understand that any system claims presented herein encompass all of the elements and limitations disclosed therein, and as such, require that each system claim be viewed as a whole. Any reasonably foreseeable items functionally related to the claims are also relevant. The Examiner, after having obtained a thorough understanding of the disclosure and claims of the present application has searched the prior art as disclosed in patents and other published documents, i.e., nonpatent literature. Therefore, as evidenced by issuance of this patent, the prior art fails to disclose or teach the elements and limitations presented in the claims as enabled by the specification and drawings, such that the presented claims are patentable under the applicable laws and rules of this jurisdiction.

Various embodiments of the present disclosure are directed to systems and techniques that provide functionality for simulating and assessing performance of real-world activities within a virtual environment by a therapy patient. The present disclosure provides for a system integrated into a practical application with meaningful limitations as an integrated system with functionality for simulating activities of daily living (ADLs) within a virtual environment that may enable a therapy patient to experience and/or improve functional independence for performing the ADLs, and may enable a therapist to assess the virtual performance of the ADLs (e.g., in the virtual environment) by the patient in order to determine whether the patient is able to perform the ADLs safely and successfully in the real-world, without exposing the therapy patient to the real-world activity and while being able to perform the simulation and/or assessment in any real-world setting (e.g., a clinical or hospital setting). In this manner, a system implemented in accordance with embodiments herein may provide an improvement to the field of therapy and clinical assessments.

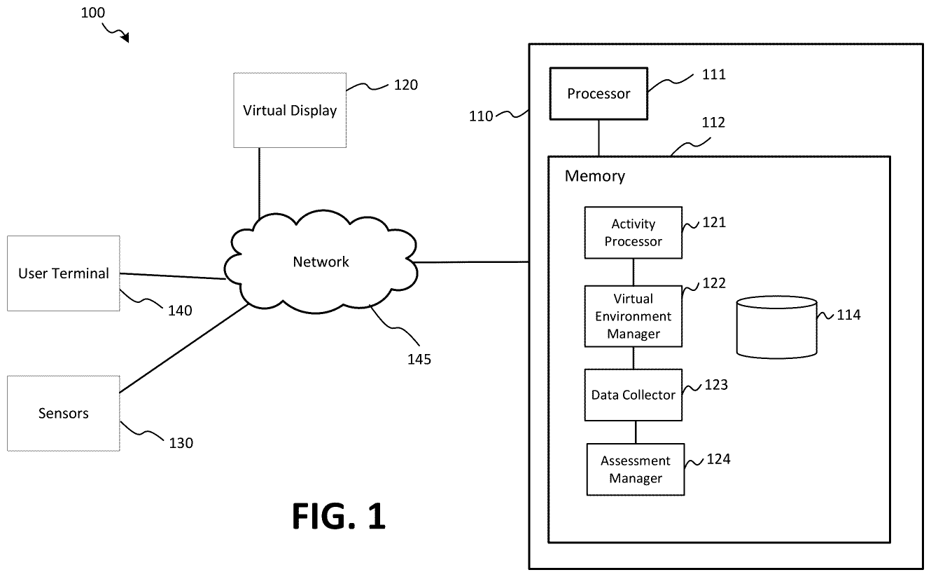

FIG. 1 is a block diagram of an exemplary system 100 configured with capabilities and functionality for simulating and assessing performance of real-world activities within a virtual environment by a therapy patient in accordance with embodiments of the present disclosure. As shown in FIG. 1, system 100 may include server 110, virtual display 120, user terminal 140, sensors 130, and network 145. These components, and their individual components, may cooperatively operate to provide functionality in accordance with the discussion herein.

It is noted that the discussion herein focuses on operations with respect to simulating and/or assessing performance of real-world activities, such as ADLs, by a therapy patient, and in particular by a therapy patient with a brain injury. In this context, embodiments of the present disclosure may allow the therapy patient to experience the performance of ADLs in a virtual environment without being exposed to dangers of the real-world activity, and may allow the therapist to assess the performance of the therapy patient without the drawbacks of real-world assessments. However, it will be appreciated that the present disclosure is not limited to such applications, and may be applicable to any situation in which it may be beneficial to allow a user to experience or learn an activity, and/or a supervisor to assess the user's performance of the activity, without actually exposing the user to the real-world activity. In this case, a system implemented in accordance with the present disclosure may provide a virtual environment configured to allow the user to learn or experience functional independence (e.g., with respect to the activity) in the virtual environment, and the supervisor to assess the user's performance of the activity (e.g., to demonstrate functional independence with respect to the activity), without exposing the user to the real-world activity and the dangers or inconveniences associated with the real-world activity. In some cases, the features of the present disclosure may be applicable in occupational therapy, physical therapy, rehabilitative therapy, and/or any other type of therapy.

The present disclosure may also be applicable for training applications. For example, a system implemented in accordance with the present disclosure may be used for ADL training, in which a therapy patient may leverage the functionality of the system to experience and train performing ADLs without exposing the therapy patient to the real-world ADL and the dangers or inconveniences associated with the real-world ADL. In another illustrative and non-limiting example, a system implemented in accordance with the present disclosure may provide functionality to train a toddler to go to the bathroom. In this example, the toddler may experience the activity and may acquire functional independence using the virtual environment, without requiring the toddler to actually perform the activity in real life. The familiarization of the toddler with the activity (e.g., from the virtual environment) may facilitate the toddler performing the real-world activity. Moreover, a guardian may be able to assess the level of functional independence of the toddler with respect to the activity (e.g., in the virtual environment) without the cumbersome requirements of the real-world activity (e.g., having to wait until the toddler actually has a need to perform the activity).

It is noted that the functional blocks, and components thereof, of system 100 of embodiments of the present invention may be implemented using processors, electronics devices, hardware devices, electronics components, logical circuits, memories, software codes, firmware codes, etc., or any combination thereof. For example, one or more functional blocks, or some portion thereof, may be implemented as discrete gate or transistor logic, discrete hardware components, or combinations thereof configured to provide logic for performing the functions described herein. Additionally, or alternatively, when implemented in software, one or more of the functional blocks, or some portion thereof, may comprise code segments operable upon a processor to provide logic for performing the functions described herein.

It is also noted that various components of system 100 are illustrated as single and separate components. However, it will be appreciated that each of the various illustrated components may be implemented as a single component (e.g., a single application, server module, etc.), may be functional components of a single component, or the functionality of these various components may be distributed over multiple devices/components. In such embodiments, the functionality of each respective component may be aggregated from the functionality of multiple modules residing in a single, or in multiple devices.

It is further noted that functionalities described with reference to each of the different functional blocks of system 100 described herein is provided for purposes of illustration, rather than by way of limitation and that functionalities described as being provided by different functional blocks may be combined into a single component or may be provided via computing resources disposed in a cloud-based environment accessible over a network, such as one of network 145.

Virtual display 120 may be configured to facilitate presentation of a virtual environment to a user. In embodiments, the virtual environment may be provided by server 110, and may be configured to enable performance of one or more target virtual tasks associated with a real-world activity, as discussed in more detail below. A user may be immersed into the virtual environment via virtual display 120. The virtual environment may be configured to facilitate virtual tasks to be performed by the user within the virtual environment. These virtual tasks may be associated with a real-world activity (e.g., with real-world physical tasks associated with the real-world activity, such as an ADL). For example, in one particular implementation, system 100, under control of server 110 cooperatively operating with virtual display 120, may generate a virtual environment in which a user (e.g., a therapy patient) may be enabled to perform one or more virtual tasks associated with an ADL (e.g., cooking a meal, doing laundry, washing dishes, going to the toilet, etc.). In embodiments, virtual display 120 may be configured to display the virtual environment in which one or more virtual tasks may be performed by the therapy patient.

In some embodiments, virtual display 120 may comprise a virtual reality (VR) device, an augmented reality device, a mixed reality device, a computer screen, a television screen, a projector, and/or any device configured to display a virtual environment. For example, virtual display 120 may be a headset, or any other VR display configured to display a virtual representation of an environment (e.g., a virtual environment).

User terminal 140 may include a mobile device, a smartphone, a tablet computing device, a personal computing device, a laptop computing device, a desktop computing device, a computer system of a vehicle, a personal digital assistant (PDA), a smart watch, another type of wired and/or wireless computing device, or any part thereof. In embodiments, user terminal 140 may be configured to provide a user interface (e.g., a graphical user interface (GUI)) structured to facilitate an operator interacting with system 100, e.g., via network 145, to execute and leverage the features provided by server 110. In embodiments, user terminal 140 may be configured to communicate with other components of system 100.

In embodiments, user terminal 140 may be configured to accept input from users, such as a patient or a therapist, that may be used to specify patient's characteristics, such as patient's limitations, affected motor functions, as well as to make selections related to the real-world activity or activities (e.g., the ADL(s)) being performed by the patient and/or being assessed by the therapist during a current session. For example, a therapist may use user terminal 140 to initiate a session, to specify or select an ADL to be performed by the patient, and to initiate the performance of the virtual tasks associated with the ADL. In embodiments, user terminal 140 may be configured to provide output. The output of user terminal 140 may include a presentation, display, or reproduction of the virtual environment presented to the therapy patient. This may enable the therapist to monitor in real-time what the patient is perceiving in the virtual environment, and to monitor the performance of the virtual tasks by the therapy patient, as well as to monitor the therapy patient's progress. In addition, the output of user terminal 140 may include reports including data associated with and/or indicative of the performance of the therapy patient with respect to the virtual tasks associated with the real-world activities.

Sensors 130 may include one or more sensors configured to measure or capture data that may include conformation, location, movement, speed, velocity, tilt, position, force, acceleration, etc., measured at a target site at which sensors 130 may be positioned on the therapy patient. The captured sensor data may be related to a particular action or movement by the therapy patient. In embodiments, the data captured by sensors 130 may be used to determine whether the therapy patient is performing a particular virtual task within normal parameters (e.g., within parameters determined to be indicate a passing performance) or performing the particular virtual task outside normal parameters (e.g., indicative of a failing performance). For example, the data captured by sensors 130 may indicate that the therapy patient has not performed a virtual task, has performed a task but has taken too long, has taken a dangerous action when performing a virtual task (e.g., has touched a hot surface, etc.), etc., indicating that the patient has failed to perform the virtual task successfully.

In embodiments, network 145 may facilitate communications between the various components of system 100 (e.g., server 110, virtual display 120, sensors 130, and/or user terminal 140). Network 145 may include a wired network, a wireless communication network, a cellular network, a cable transmission system, a Local Area Network (LAN), a Wireless LAN (WLAN), a Metropolitan Area Network (MAN), a Wide Area Network (WAN), the Internet, the Public Switched Telephone Network (PSTN), a Bluetooth connection, a radio frequency (RF) connection, etc.

Server 110 may be configured to, in operation according to embodiments, represent a real-world activity (e.g., an ADL) as a plurality of target virtual tasks (e.g., a plurality of target virtual tasks corresponding to a plurality of target physical tasks associated with the real-world activity), generate a virtual environment configured to enable performance of the plurality of target virtual tasks, receive data indicative of performance of each target virtual task by the therapy patient, and to determine, based on the data indicative of the performance of each target virtual task by the therapy patient, whether the therapy patient is able to perform the real-world activity successfully. In this manner, the functionality of server 110 may enable a therapy patient to improve functional independence by providing a mechanism for the therapy patient to experience or learn functional independence with respect to real-world ADLs in a virtual environment, and may enable a therapist to assess the virtual performance associated with ADLs by the patient in order to determine whether the patient is able to perform the ADLs safely and successfully in the real-world, without exposing the therapy patient to the real-world ADLs and while being able to perform the simulation and/or assessment in any real-world setting (e.g., a clinical or hospital setting) without constraints to a particular location. The functionality of server 110 may be provided by the cooperative operation of various components of server 110, as will be described in more detail below.

Although FIG. 1 shows a single server 110, it will be appreciated that server 110 and its individual functional blocks may be implemented as a single device or may be distributed over multiple devices having their own processing resources, whose aggregate functionality may be configured to perform operations in accordance with the present disclosure. Furthermore, those of skill in the art would recognize that although FIG. 1 illustrates components of server 110 as single blocks, the implementation of the components and of server 110 is not limited to a single component and as described above, may be distributed over several devices or components.

It is noted that the various components of server 110 are illustrated as single and separate components in FIG. 1. However, it will be appreciated that each of the various components of server 110 may be a single component (e.g., a single application, server module, etc.), may be functional components of a same component, or the functionality may be distributed over multiple devices/components. In such embodiments, the functionality of each respective component may be aggregated from the functionality of multiple modules residing in a single, or in multiple devices.

As shown in FIG. 1, server 110 includes processor 111 and memory 112. Processor 111 may comprise a processor, a microprocessor, a controller, a microcontroller, a plurality of microprocessors, an application-specific integrated circuit (ASIC), an application-specific standard product (ASSP), or any combination thereof, and may be configured to execute instructions to perform operations in accordance with the disclosure herein. In some embodiments, as noted above, implementations of processor 111 may comprise code segments (e.g., software, firmware, and/or hardware logic) executable in hardware, such as a processor, to perform the tasks and functions described herein. In yet other embodiments, processor 111 may be implemented as a combination of hardware and software. Processor 111 may be communicatively coupled to memory 112.

As shown in FIG. 1, memory 112 includes activity processor 121, virtual environment manager 122, data collector 123, assessment manager 124, and database 114. Memory 112 may comprise one or more semiconductor memory devices, read only memory (ROM) devices, random access memory (RAM) devices, one or more hard disk drives (HDDs), flash memory devices, solid state drives (SSDs), erasable ROM (EROM), compact disk ROM (CD-ROM), optical disks, other devices configured to store data in a persistent or non-persistent state, network memory, cloud memory, local memory, or a combination of different memory devices. Memory 112 may comprise a processor readable medium configured to store one or more instruction sets (e.g., software, firmware, etc.) which, when executed by a processor (e.g., one or more processors of processor 111), perform tasks and functions as described herein.

In embodiments, memory 112 may be configured to facilitate storage operations. For example, memory 112 may include database 114 configured to store data related to operations of server 110, such as therapy patient-related information, system configuration information, task passing or failing thresholds, AI-models for generating virtual tasks associated with a real-world activity, etc. In some embodiments, database 114 may be integrated into memory 112, or may be provided as a separate module. In yet other embodiments, database 114 may be a single database, or may be a distributed database implemented over a plurality of database modules. Database 114 may be configured to store information for a plurality of patients. In some cases, the information may be used in training and learning operations.

Activity processor 121 may be configured to analyze and/or process one or more real-world activities, and to represent the one or more real-world activities as a plurality of virtual tasks that may be performed within a virtual environment. Within the context of the present disclosure, the one or more real-world activities may include one or more ADLs. As mentioned above, a therapy patient may currently be under restrictions, such as medical restrictions. The medical restrictions may include the therapy patient being limited or prevented from performing the one or more activities, or may be under supervised care and not allowed to perform one or more ADLs unsupervised. In embodiments, the one or more real-world activities may represent activities that may be determined to be indicative of functional independence. For example, the one or more real-world activities may include activities that, when performed successfully and within normal parameters by the therapy patient, may indicate that the therapy patient is capable of functional independence, and may indicate to a therapist or medical professional that the current medical restrictions on the therapy patient may be removed or at least loosened. For example, the one or more real-world activities may include activities such as going to the toilet, dressing, personal grooming, cooking, doing the laundry, doing the dishes, sweeping and/or mopping the floors, other household chores, etc. Of course, it should be appreciated that the above list of real-world activities is provided for illustrative purposes and should not be construed as limiting in any way. Indeed, other activities may be included in the real-world activities described herein.

In embodiments, activity processor 121 may be configured to represent a real-world activity as a plurality of target virtual tasks. In this case, the plurality of target virtual tasks may be configured to be indicative of performance of the real-world activity. For example, the plurality of target virtual tasks may be configured such that successful performance of the plurality of target virtual tasks by the therapy patient indicates a likelihood that the therapy patient may successfully perform the real-world activity. In this manner, successful performance of the plurality of target virtual tasks may be indicative of successful performance of the associated real-world activity (e.g., the real-world activity represented by the plurality of target virtual tasks).

In embodiments, activity processor 121 may be configured to represent a real-world activity as a plurality of target virtual tasks based on an analysis of the real-world activity. The analysis of the real-world activity may include determining and/or identifying (e.g., manually and/or based on a trained machine learning (ML) or artificial intelligence (AI) model) the plurality of target virtual tasks that may be indicative of successful performance of the real-world activity.

In some embodiments, the plurality of target virtual tasks representing a real-world activity may be unrelated to the actual tasks of the real-world activity. For example, the real-world activity may include an ADL of sweeping a floor. In this example, the plurality of target virtual tasks representing the floor mapping real-world activity may include virtual tasks associated with a hockey game in the virtual environment, which, although unrelated to sweeping a floor, may be indicative of a patient's ability to sweep floors. These embodiments may be especially useful when a single set of virtual tasks may be indicative of real-world performance of several real-world activities (e.g., virtual hockey game may be indicative of real-world ability to sweep and/or mop floors, rake the yard, etc.).

In embodiments, the configuration of activity processor 121 to represent a real-world activity as a plurality of target virtual tasks may include configuration of activity processor 121 to represent the real-world activity as a plurality of target physical tasks, and to generate the plurality of target virtual tasks, where each target virtual task of the plurality of target virtual tasks is associated with a target physical task of the plurality of target physical tasks such that a performance of a target virtual task is indicative of performance of a respective target physical task. For example, activity processor 121 may determine or identify (e.g., manually by a user or automatically using a trained ML or AI model) a plurality of target physical tasks that, in the aggregate, may represent the real-world activity. The target physical tasks may represent discrete real-world tasks that, in the aggregate, represent the real-world activity. In this case, performing the real-world activity may be accomplished by performing each of the plurality of target physical tasks. Based on the plurality of target physical tasks associated with the real-world activity, activity processor 121 may generate the plurality of target virtual tasks.

For example, as a non-limiting example, an ADL may include cooking a piece of meat in the oven. In this example, activity processor 121 may analyze the real-world activity of cooking a piece of meat in the oven, and may represent the real-world activity as a plurality of target physical tasks. In this example, activity processor 121 may determine that the ADL of cooking a piece of meat in the oven may be represented by the target physical tasks including, among others, turning on the oven, retrieving the meat from the fridge, closing the fridge door, placing the meat in the pan, placing the pan in the oven, closing the oven door, monitoring the meat until cooked, putting on oven mitts, removing the pan from the oven, closing the oven door, and turning off the oven. In embodiments, activity processor 121 may determine that the ADL of cooking a piece of meat in the oven may be represented by target physical tasks that may include more less, and/or other physical tasks, and the above list is not intended to be limiting in any way. In this example, activity processor 121 may generate a plurality of target virtual tasks that may include a target virtual task corresponding to each of the target physical tasks representing the ADL of cooking a piece of meat in the oven. In this case, activity processor 121 may generate a target virtual task A corresponding to turning on the oven, a target virtual task B corresponding to retrieving the meat from the fridge, and so on to virtual task N corresponding to turning off the oven, where a target virtual task is generated for each target physical task of the plurality of target physical tasks representing the ADL of cooking a piece of meat in the oven.

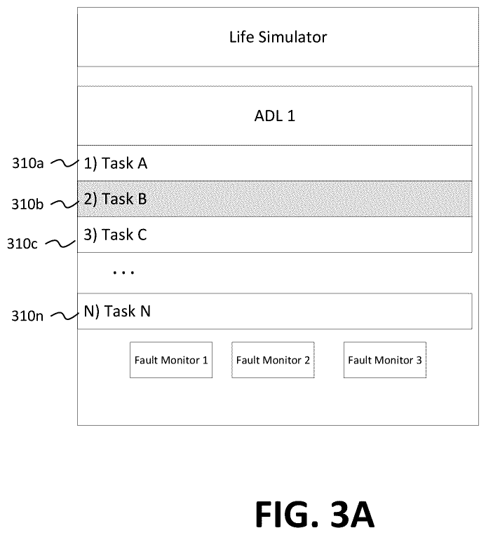

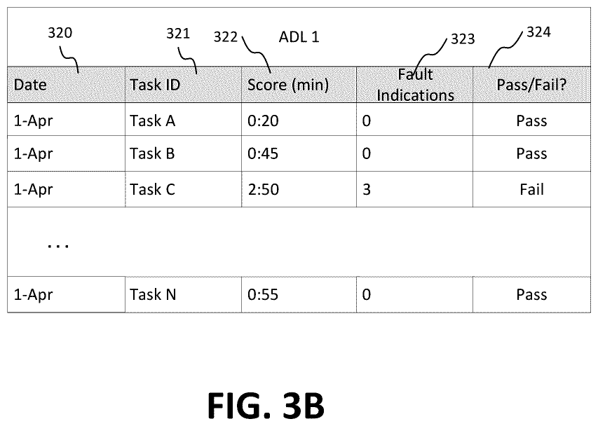

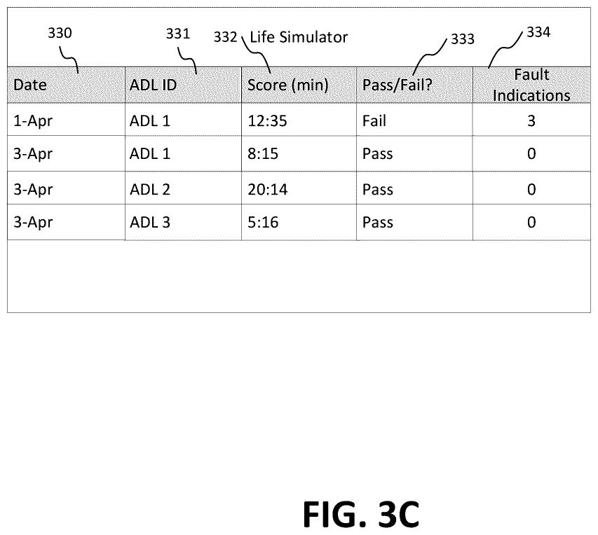

FIG. 3A shows an example of a plurality of target virtual tasks generated to represent a real-world activity in accordance with embodiments of the present disclosure. In the example illustrated in FIG. 3A, real-world activity ADL 1 may be represented as a plurality of target virtual tasks including target virtual task A-target virtual task N.

With reference back to FIG. 1, a target virtual task may represent a task that may be performed (e.g., by the therapy patient) within a virtual environment. For example, following the example above of the ADL including cooking a piece of meat in the oven, each of the target virtual tasks of the plurality of target virtual tasks corresponding to the plurality of target physical tasks representing the ADL of cooking a piece of meat in the oven may represent a virtual task that is to be performed within a virtual environment, rather than in the real-world. In this manner, the therapy patient may experience performance of the real-world ADL of cooking a piece of meat in the oven without actually having to physically cook a real piece of meat in a real oven, which may be dangerous and inconvenient in a clinical setting.

In embodiments, activity processor 121 may generate the plurality of target virtual tasks as a sequential series of target virtual tasks. In these embodiments, each target virtual task in the sequential series of target virtual tasks follows another in sequence. In some embodiments, the therapy patient may be prevented from performing a second target virtual task until a first target virtual task is performed or completed, when the second target virtual task follows the first target virtual task in the sequential series of target virtual tasks. For example, continuing to follow the example above of the ADL including cooking a piece of meat in the oven, a second target virtual task, which may correspond to the target physical task of placing the pan in the oven may follow a first target virtual task, which may correspond to the target physical task of placing the meat in the pan. In this case, the therapy patient may be prevented from placing the pan in the oven until the therapy patient has placed the meat in the pan within the virtual environment. In this manner, activity processor 121 may ensure that the therapy patient experiences the ADL including cooking a piece of meat in the oven in a guided manner, which may allow the therapy patient to learn the proper sequence of tasks for performing the ADL.

In additional or alternative embodiments, activity processor 121 may generate the plurality of target virtual tasks as a non-sequential series of target virtual tasks. In these embodiments, each target virtual task in the non-sequential series of target virtual tasks is allowed to be performed by the therapy patient notwithstanding performance of other target virtual tasks of the non-sequential series of target virtual tasks. For example, the therapy patient may be allowed to perform a second target virtual task whether or not a first target virtual task is performed or completed. For example, continuing to follow the example above of the ADL including cooking a piece of meat in the oven, the therapy patient may be allowed to perform a second target virtual task, which may correspond to the target physical task of placing the pan in the oven, whether or not the therapy patient has performed a first target virtual task, which may correspond to the target physical task of placing the meat in the pan. These configurations may allow the therapist to detect or ascertain situation in which the therapy patient may conduct an activity in a sequence of tasks that may not make sense or be advisable, and in this manner, the therapist may determine that the therapy patient is not able to perform the ADL.

Virtual environment manager 122 may be configured to generate a virtual environment configured to enable performance of the plurality of target virtual tasks representing the real-world activity. In embodiments, virtual environment manager 122 may generate a virtual environment that is presented to the therapy patient (e.g., via virtual display 120) and that may allow the therapy patient to interact with the virtual environment in such a way as to enable the therapy patient to perform each of the target virtual tasks representing the real-world activity. The configuration of the virtual environment may be previously set by operations requirements, or may be dynamically generated based on the patient characteristics, physical location of the therapy session and/or assessment, etc.

For example, continuing to follow the example above of the ADL including cooking a piece of meat in the oven, virtual environment manager 122 may generate a virtual environment that may include a virtual representation of a kitchen, with a virtual representation of an oven, a fridge, a pan, and a piece of meat. In this manner, the therapy patient may, during operation, be immersed in the virtual environment and may be enabled to perform the target virtual tasks (e.g., target virtual task A-target virtual task N) corresponding to the target physical tasks representing the ADL including cooking a piece of meat in the oven.

In some embodiments, virtual environment manager 122 may be configured to vary the layout of the virtual environment session over session. For example, virtual environment manager 122 may generate a virtual environment with a first layout for a first session, and may generate a virtual environment with a second layout, different from the first layout, for a second session. These functionality may facilitate ensuring that the therapy patient does not grow accustomed to a particular layout and “memorizes” the layout, such that an assessment of the therapy patient's ability to perform a real-world activity may be compromised. In some embodiments, similar functionality may be provided to activity processor 121 to vary the plurality of target virtual tasks to represent a real-world activity to ensure that a therapy patient does not memorize the sequence and may provide a better assessment of the therapy patient's ability to perform a real-world activity.

In embodiments, virtual environment manager 122 may generate a stock virtual environment. For example, virtual environment manager 122 may generate a virtual kitchen environment that is unrelated to the therapy patient's home environment (e.g., the therapy patient's own kitchen or a kitchen familiar to the therapy patient). In these embodiments, the virtual kitchen environment generated by virtual environment manager 122 may not accurately reflect the therapy patient's actual home setting.

In alternative or additional environments, virtual environment manager 122 may generate a virtual environment manager 122 that is actually related to the therapy patient's home environment or surrounding (e.g., a surrounding associated with the therapy patient). For example, in some embodiments, a home environment familiar to the therapy patient, or a home environment in which the therapy patient is to perform real-world activities, may be mapped, and the map may be used by virtual environment manager 122 to generate the virtual environment. These embodiments may provide the advantage of allowing the therapy patient to experience real-world activities (e.g., such as ADLs), and allowing the therapist to evaluate the therapy patient's performance of ADLs, in a simulated or virtual environment that may be very close to the actual real-world environment in which the therapy patient will be performing the ADLs.

In embodiments, the configuration of virtual environment manager 122 to generate the virtual environment may include configuration of virtual environment manager 122 to configure at least one virtual fault monitor. A virtual fault monitor may include functionality to detect a deviation from the normal performance parameters during performance of a target virtual task, and to generate a fault indication in response to detecting the deviation from the normal performance parameters. In embodiments, the deviation from the normal performance parameters during performance of a target virtual task may include deviations that may represent a dangerous, unsafe, or severe deviation. For example, continuing to follow the example above of the ADL including cooking a piece of meat in the oven, a deviation from the normal performance parameters during performance of the target virtual tasks associated with the ADL of cooking a piece of meat in the oven may include touching a hot surface of the oven, touching the hot pan after cooking without putting on the mitts, leaving the fridge door open, etc. In embodiments, the fault indication may include a severity level indicating how far out of normal performance parameters the deviation is. For example, when the virtual fault monitor monitors whether a hot surface has been touched, the fault indication generated by the virtual fault monitor may include an indication of the severity of the burn that the therapy patient has suffered (e.g., virtually, of course) based, for example, on the length of time the hot surface was touched, the contact area with the hot surface, the virtual temperature of the hot surface, etc.

In embodiments, upon detection of a deviation from the normal performance parameters, the virtual fault monitor may generate an alert to notify the therapy patient of the deviation. In some embodiments, such as during assessments, the virtual fault monitor may keep track of any fault indications generated, which may be reported to the therapists and/or may be considered when determining whether the therapy patient has passed or failed a target virtual tasks and/or a real-world activity.

In embodiments, the configuration of virtual environment manager 122 to generate the virtual environment may include configuration of virtual environment manager 122 to configure the virtual environment to allow the therapy patient to multitask. For example, virtual environment manager 122 may configure the virtual environment to allow the therapy patient to perform target virtual tasks associated with the real-world activity, but also to allow the therapy patient to perform target virtual tasks associated with another real-world activity. For example, some target virtual tasks may include wait times (e.g., a target virtual task of waiting for the piece of meat to cook may include a cooking time). The wait time may be simulated within the virtual environment as a real-time wait time, although in some configurations the therapy patient may be allowed to fast forward through the wait time such that the therapy patient does not have to wait in real-time. In these cases, while the therapy patient is waiting for the wait time to complete, the therapy patient may be allowed to perform other target virtual tasks associated with other rea-world activities. For example, in the meat cooking example above, while the therapy patient is waiting for the meat to cook in the oven, the therapy patient may be allowed to drop a load of laundry in a virtual washing machine. In this manner, the therapy patient may multi-task within the virtual environment.

The functionality of system 100 to simulate and assess multitasking by a therapy patient is significant, as it may allow a therapy patient to experience and practice, and a therapist to evaluate, the therapy patient's ability to perform various activities while being able to keep track of the various tasks associated with the different activities. For example, the therapist may be able to assess the ability of the therapy patient to remember and/or keep track of where the therapy patient is with respect to an activity after the therapy patient may have performed virtual tasks associated with a different activity. For example, in the meat cooking example above, by allowing the therapy patient to drop a load of laundry in a virtual washing machine while the therapy patient is waiting for the meat to cook in the oven, the therapist may be able to assess whether the therapy patient is able to keep track, or remember, that the meat is in the virtual oven, or whether the therapy patient is not able to remember that. This multitasking functionality is a powerful tool to assess the cognitive abilities of a therapy patient.

Data collector 123 may be configured to receive data indicative of performance of each target virtual task of the plurality of target virtual tasks by the therapy patient with the brain injury. In embodiments, the data indicative of performance of each target virtual task of the plurality of target virtual tasks may include data collected (e.g., by sensors 130 and/or virtual display 120) during performance of the target virtual tasks. In embodiments, the data indicative of performance of each target virtual task of the plurality of target virtual tasks may include a time taken by the therapy patient to complete each target virtual task of the plurality of target virtual tasks. The time taken to complete a target virtual task may be measured based on a start time for the target virtual task, such as a time at which the target virtual task is started to be performed by the therapy patient, is started by the therapist (e.g., via user terminal 140) whether or not the therapy patient has started to perform the target virtual task, and/or a time at which the virtual tasks is supposed to be started (e.g., based on configuration or based on a preceding target virtual task being completed), and an end time for the target virtual task, such as a time at which the target virtual task is completed by the therapy patient and/or is marked as completed by the therapist (e.g., via user terminal 140).

In embodiments, the data indicative of performance of each target virtual task of the plurality of target virtual tasks may include a number of fault indications occurring during performance of each target virtual task. For example, fault indications generated by virtual fault monitors for a target virtual task (e.g., occurring during performance of the target virtual task) may be reported as part of the data indicative of performance of the target virtual task. As mentioned herein, a fault indication for a target virtual task may indicate a deviation from normal performance parameters by the therapy patient during performance of the target virtual task.

Assessment manager 124 may be configured to determining, based on the data indicative of the performance of each target virtual task of the plurality of target virtual tasks by the therapy patient, whether the therapy patient is able to perform the real-world activity successfully or not. In embodiments, the therapist may analyze the results of each individual target virtual task (e.g., based on the data indicative of performance of each target virtual task of the plurality of target virtual tasks), and/or the results of the target virtual tasks in the aggregate, and may determine whether the therapy patient is able to perform the real-world activity associated with the target virtual tasks safely and/or effectively. In this manner, the therapist may determine whether the therapy patient is capable of functional independence based on the results. In some embodiments, the results of the individual target virtual task and/or the results of the target virtual tasks in the aggregate may be determined by the therapist manually and/or by assessment manager 124 automatically.

In embodiments, the performance of each target virtual task of the plurality of target virtual tasks may be rated as either pass or fail. A passing performance of a target virtual task may indicate that the target virtual task was completed successfully and within acceptable parameters. In some embodiments, a passing performance may also indicate that there is a high likelihood that a target physical task corresponding to the passing performance can be performed successfully and within acceptable parameters by the therapy patient. On the other hand, a failing performance of a target virtual task may indicate that the target virtual task was not completed successfully or was performed outside of acceptable parameters. In some embodiments, a failing performance may also indicate that there is a high likelihood that a target physical task corresponding to the failing performance cannot be performed successfully and within acceptable parameters by the therapy patient.

In embodiments, each target virtual task may be associated with a score. For example, the performance of a target virtual task may be scored while the therapy patient is performing the target virtual task. The target virtual task score may include a time taken to perform the target virtual task. In some embodiments, the time taken to perform the target virtual task may be compared against a passing threshold configured for the target virtual task. In this case, if the time taken to perform the target virtual task exceeds the passing threshold, the performance of the target virtual task may be rated as a failing performance. On the other hand, if the time taken to perform the target virtual task does not exceed the passing threshold, the performance of the target virtual task may be rated as a passing performance.

In an additional or alternative example, the target virtual task score may include a number of fault indications that occur during performance of the target virtual task. In some embodiments, the number of fault indications that occur during performance of the target virtual task may be compared against a fault threshold configured for the target virtual task. In this case, if the number of fault indications that occur during performance of the target virtual task exceeds the fault threshold, the performance of the target virtual task may be rated as a failing performance. On the other hand, if the number of fault indications that occur during performance of the target virtual task does not exceed the fault threshold, the performance of the target virtual task may be rated as a passing performance.

In embodiments, determining whether the therapy patient is able to perform the real-world activity associated with the target virtual tasks safely and/or effectively may include determining whether each target virtual task of the plurality of target virtual tasks has a passing performance or not. For example, the therapist may determine that the therapy patient is able to perform the real-world activity associated with the target virtual tasks safely and/or effectively in response to a determination that all target virtual tasks have a passing performance score. In another example, the therapist may determine that the therapy patient is unable to perform the real-world activity associated with the target virtual tasks safely and/or effectively in response to a determination that at least one target virtual task has a failing performance score. In some embodiments, the therapist may determine that the therapy patient is able to perform the real-world activity associated with the target virtual tasks safely and/or effectively in response to a determination that a threshold number of target virtual tasks have a passing performance score (e.g., the number of target virtual tasks with a passing performance score exceed the threshold number), or may determine that the therapy patient is unable to perform the real-world activity associated with the target virtual tasks safely and/or effectively in response to a determination that the number of target virtual tasks with a passing performance score does not exceed the threshold number.

In some embodiments, determining whether the therapy patient is able to perform the real-world activity associated with the target virtual tasks safely and/or effectively may include aggregating the individual performance scores of each target virtual task of the plurality of target virtual tasks into an overall activity score, and determining that the overall activity score is above a passing threshold. In embodiments, aggregating the individual performance scores may include adding or averaging the individual performance scores. In these embodiments, in response to a determination that the overall activity score is above the passing threshold, the therapist may determine that the therapy patient is able to perform the real-world activity associated with the target virtual tasks safely and/or effectively. On the other hand, in response to a determination that the overall activity score is not above the passing threshold, the therapist may determine that the therapy patient is unable to perform the real-world activity associated with the target virtual tasks safely and/or effectively.

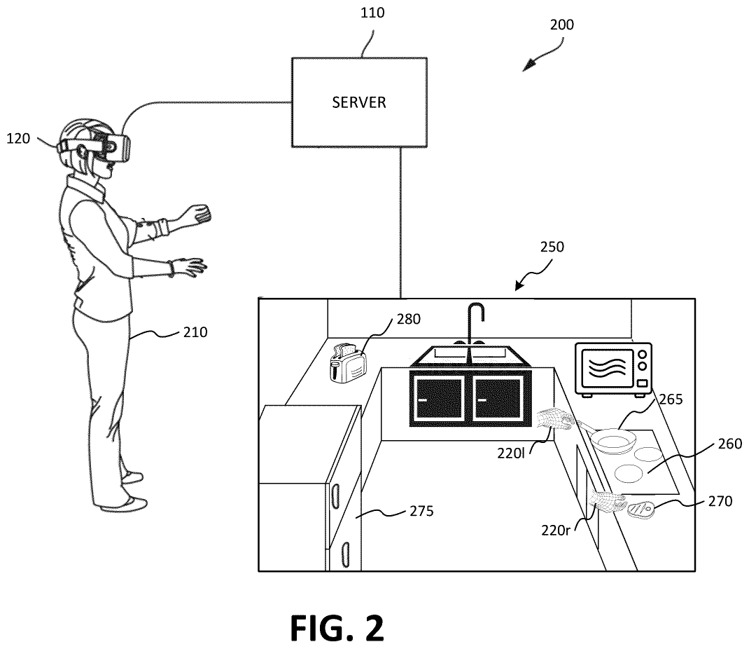

FIG. 2 illustrates an example embodiment implemented in system 200 in accordance with aspects of the present disclosure for providing functionality as discussed herein. In this example, during operation, a therapy patient 210 may be connected into system 200. In this example, the therapy patient 210 may be under medical restrictions limiting the therapy patient 210's functional independence. The therapy patient 210 may be connected to system 200 by placing virtual display 120 upon the therapy patient 210's head. Virtual display 120 may configured to display the virtual environment 250, as illustrated in FIG. 2, into which the therapy patient 210 has been immersed. In this example, which includes a specific and non-limiting example of simulating and assessing an ADL including cooking a piece of meat upon a stove, the virtual environment simulates a kitchen environment. As illustrated, the virtual kitchen environment may include a virtual representation of stove 260, pan 265, meat 270, fridge 275, and toaster 280. In this example, the therapy patient 210's hands may be represented by virtual right hand 220r and virtual left hand 2201.

The virtual environment in this example may be configured to enable the therapy patient to perform the ADL of cooking a steak over a stove. In embodiments, the ADL of cooking a steak over a stove may be represented (e.g., using functionality of an activity processor such as activity processor 121) as a plurality of virtual tasks. For example, FIG. 3A may illustrate the representation of ADL 1 (e.g., as presented to the therapist via a user terminal, such as user terminal 140), which in this example may include the ADL of cooking a steak over a stove, as a plurality of virtual tasks including virtual task 310a-310n.

In embodiments, the virtual environment may also include one or more virtual fault monitors 1-3. As described herein, the virtual fault monitors may include functionality to detect deviations from the normal performance parameters during performance of the target virtual tasks associated with the ADL of cooking a steak over a stove, and to generate a fault indication in response to detecting a deviation from the normal performance parameters. In embodiments, the virtual fault monitors may be activated by the therapist by selecting a respective fault monitor 1-3, as desired.

With reference back to FIG. 2, the therapy patient 210 may perform the plurality of virtual tasks associated with the ADL of cooking a steak over a stove. During performance of the plurality of virtual tasks, data indicative of the performance of each of the plurality of virtual tasks may be collected and provided to server 110. In embodiments, upon completion of the plurality of virtual tasks, the data indicative of the performance of each of the plurality of virtual tasks may be reported. FIG. 3B illustrates a report including data indicative of performance of virtual tasks of a plurality of virtual tasks associated with a real-world activity in accordance with embodiments of the present disclosure. As shown in FIG. 3B, the data indicative of the performance of each of the plurality of virtual tasks task A-task N associated with real-world ADL 1 (e.g., the ADL of cooking a steak over a stove) may include a date indication 321, and a task ID 321 to identify the respective tasks. The data indicative of the performance may also include a score 322 for each of the virtual tasks, and fault indication count 323 for each of the virtual tasks to indicate the number of fault indications that occurred during the performance of each of the virtual tasks. In optional embodiments, the data indicative of the performance may also include a pass/fail indication 324 to indicate whether a particular virtual task was performed with a passing or failing performance by the therapy patient. Whether a particular virtual task was performed with a passing or failing performance by the therapy patient may be determined in accordance with the functionality as described above with respect to FIG. 1. As shown in this example, based on the performance by therapy patient 210 on 1-April, the therapy patient performed virtual task C with a failing performance, and the remaining of virtual task A-virtual task N with a passing performance.

In embodiments, a report may also be generated to include data related to the overall performance of the ADL. For example, FIG. 3C illustrates a report including data related to the overall performance of real-world activities in accordance with embodiments of the present disclosure. As shown in FIG. 3C, the report may include a date indication 330 of when the activity was evaluated or experienced, and an ADL ID 331 to identify the respective activity being performed or evaluated. The report may also include a score 332 for each of the activities, which may represent an aggregated score of each of the individual scores of the virtual tasks associated with each activity, and fault indication count 333 to indicate the number of fault indications that occurred during the performance of the virtual tasks associated with each respective activity. In optional embodiments, the report may also include a pass/fail indication 334 to indicate whether the performance of the therapy patient indicates that the therapy patient is likely to perform the activity safely and effectively. Whether the performance of the therapy patient indicates that the therapy patient is likely to perform the activity safely and effectively may be determined in accordance with the functionality as described above with respect to FIG. 1. As shown in this example, based on the performance by therapy patient 210 on 1-April, it is determined that it is highly likely that the therapy patient 210 is unable to perform the real-world ADL 1 (e.g., the ADL of cooking a steak over a stove) safely and effectively. However, as also shown in FIG. 3C, based on the performance by therapy patient 210 on 3-April, it is determined that it is highly likely that the therapy patient 210 is able to perform the real-world ADL 1 (e.g., the ADL of cooking a steak over a stove), as well as real-world ADLs 2 and 3, safely and effectively.

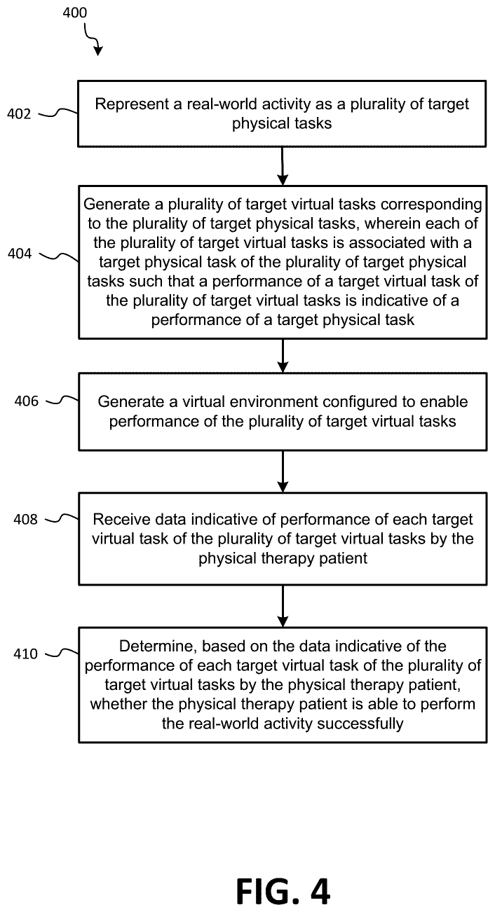

FIG. 4 shows a high-level flow diagram 400 of operation of a system configured for providing functionality for simulating and assessing performance of real-world activities within a virtual environment by a therapy patient in accordance with embodiments of the present disclosure. For example, the functions illustrated in the example blocks shown in FIG. 4 may be performed by system 100 of FIG. 1 according to embodiments herein. In embodiments, the operations of the method 400 may be stored as instructions that, when executed by one or more processors, cause the one or more processors to perform the operations of the method 400.

At block 402, a real-world activity is represented as a plurality of target physical tasks. In embodiments, functionality of an activity processor (e.g., activity processor 121 as shown in FIG. 1) may be used to represent a real-world activity as a plurality of target physical tasks. In embodiments, the activity processor may perform operations to represent a real-world activity as a plurality of target physical tasks according to operations and functionality as described above with reference to activity processor 121 and as illustrated in FIG. 1-3C.

At block 404, a plurality of target virtual tasks corresponding to the plurality of target physical tasks is generated. In embodiments, each of the plurality of target virtual tasks may be associated with a target physical task of the plurality of target physical tasks such that a performance of a target virtual task of the plurality of target virtual tasks is indicative of a performance of a target physical task. In embodiments, functionality of an activity processor (e.g., activity processor 121 as shown in FIG. 1) may be used to generate a plurality of target virtual tasks corresponding to the plurality of target physical tasks. In embodiments, the activity processor may perform operations to generate a plurality of target virtual tasks corresponding to the plurality of target physical tasks according to operations and functionality as described above with reference to activity processor 121 and as illustrated in FIG. 1-3C.

At block 406, a virtual environment configured to enable performance of the plurality of target virtual tasks is generated. In embodiments, functionality of a virtual environment manager (e.g., virtual environment manger 122 as shown in FIG. 1) may be used to generate a virtual environment configured to enable performance of the plurality of target virtual tasks. In embodiments, the virtual environment manger may perform operations to generate a virtual environment configured to enable performance of the plurality of target virtual tasks according to operations and functionality as described above with reference to virtual environment manger 122 and as illustrated in FIG. 1-3C.

At block 408, data indicative of performance of each target virtual task of the plurality of target virtual tasks by the therapy patient is received. In embodiments, functionality of a data collector (e.g., data collector 123 as shown in FIG. 1) may be used to receive data indicative of performance of each target virtual task of the plurality of target virtual tasks by the therapy patient. In embodiments, the data collector may perform operations to receive data indicative of performance of each target virtual task of the plurality of target virtual tasks by the therapy patient according to operations and functionality as described above with reference to data collector 123 and as illustrated in FIG. 1-3C.

At block 410, whether the therapy patient is able to perform the real-world activity successfully is determined based on the data indicative of the performance of each target virtual task of the plurality of target virtual tasks by the therapy patient. In embodiments, functionality of an assessment manger (e.g., assessment manger 124 as shown in FIG. 1) may be used to determine, based on the data indicative of the performance of each target virtual task of the plurality of target virtual tasks by the therapy patient, whether the therapy patient is able to perform the real-world activity successfully. In embodiments, the assessment manger may perform operations to determine, based on the data indicative of the performance of each target virtual task of the plurality of target virtual tasks by the therapy patient, whether the therapy patient is able to perform the real-world activity successfully according to operations and functionality as described above with reference to assessment manger 124 and as illustrated in FIG. 1-3C.

Persons skilled in the art will readily understand that advantages and objectives described above would not be possible without the particular combination of computer hardware and other structural components and mechanisms assembled in this inventive system and described herein. Additionally, the algorithms, methods, and processes disclosed herein improve and transform any general-purpose computer or processor disclosed in this specification and drawings into a special purpose computer programmed to perform the disclosed algorithms, methods, and processes to achieve the aforementioned functionality, advantages, and objectives. It will be further understood that a variety of programming tools, known to persons skilled in the art, are available for generating and implementing the features and operations described in the foregoing. Moreover, the particular choice of programming tool(s) may be governed by the specific objectives and constraints placed on the implementation selected for realizing the concepts set forth herein and in the appended claims.

The description in this patent document should not be read as implying that any particular element, step, or function can be an essential or critical element that must be included in the claim scope. Also, none of the claims can be intended to invoke 35 U.S.C. § 112(f) with respect to any of the appended claims or claim elements unless the exact words “means for” or “step for” are explicitly used in the particular claim, followed by a participle phrase identifying a function. Use of terms such as (but not limited to) “mechanism,” “module,” “device,” “unit,” “component,” “element,” “member,” “apparatus,” “machine,” “system,” “processor,” “processing device,” or “controller” within a claim can be understood and intended to refer to structures known to those skilled in the relevant art, as further modified or enhanced by the features of the claims themselves, and can be not intended to invoke 35 U.S.C. § 112(f). Even under the broadest reasonable interpretation, in light of this paragraph of this specification, the claims are not intended to invoke 35 U.S.C. § 112(f) absent the specific language described above.

The disclosure may be embodied in other specific forms without departing from the spirit or essential characteristics thereof. For example, each of the new structures described herein, may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the inventions can be established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

Those of skill in the art would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various embodiments of the present disclosure may be combined or performed in ways other than those illustrated and described herein.

Functional blocks and modules in FIGS. 1-4 may comprise processors, electronics devices, hardware devices, electronics components, logical circuits, memories, software codes, firmware codes, etc., or any combination thereof. Consistent with the foregoing, various illustrative logical blocks, modules, and circuits described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the disclosure herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal, base station, a sensor, or any other communication device. In the alternative, the processor and the storage medium may reside as discrete components in a user terminal.

In one or more exemplary designs, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. Computer-readable storage media may be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, a connection may be properly termed a computer-readable medium. For example, if the software is transmitted from a website, server, or other remote source using a coaxial cable, fiber optic cable, twisted pair, or digital subscriber line (DSL), then the coaxial cable, fiber optic cable, twisted pair, or DSL, are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods, and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of simulating and assessing performance of real-world activities within a virtual environment by a therapy patient, comprising:
representing a real-world activity as a plurality of target physical tasks;
generating a plurality of target virtual tasks corresponding to the plurality of target physical tasks, wherein each of the plurality of target virtual tasks is associated with a target physical task of the plurality of target physical tasks such that a performance of a target virtual task of the plurality of target virtual tasks is indicative of a performance of a target physical task;
generating a virtual environment configured to enable performance of the plurality of target virtual tasks;
receiving data indicative of performance of each target virtual task of the plurality of target virtual tasks by the therapy patient; and
determining, based on the data indicative of the performance of each target virtual task of the plurality of target virtual tasks by the therapy patient, whether the therapy patient is able to perform the real-world activity successfully.

2. The method of claim 1, wherein performance of a first target virtual task of the plurality of target virtual tasks includes a wait period during which the therapy patient is to wait for completion of the first target virtual task without input by the therapy patient, and wherein generating the virtual environment configured to enable performance of the first target virtual task includes:
enabling performance of at least one target virtual task associated with another real-world activity different from the real-world activity during the wait period of the first target virtual task.

3. The method of claim 1, wherein generating the plurality of target virtual tasks corresponding to the plurality of target physical tasks includes one or more of:
generating the plurality of target virtual tasks as a sequential series of target virtual tasks, wherein a second target virtual task of the sequential series of target virtual tasks is prevented from being performed by the therapy patient until performance of a first target virtual task of the sequential series of target virtual tasks; and
generating the plurality of target virtual tasks as a non-sequential series of target virtual tasks, wherein each target virtual task of the non-sequential series of target virtual tasks is allowed to be performed by the therapy patient notwithstanding performance of other target virtual tasks of the non-sequential series of target virtual tasks.

4. The method of claim 1, wherein performance of each target virtual task of the plurality of target virtual tasks is pass or fail, wherein a passing performance of a target virtual task indicates successful completion of an associated target physical task by the therapy patient, and wherein a failing performance of the target virtual task indicates unsuccessful completion of the associated target physical task by the therapy patient.

5. The method of claim 4, wherein determining that the therapy patient is able to perform the real-world activity successfully includes one or more of:
determining that each target virtual task of the plurality of target virtual tasks has a passing performance; and
determining that a threshold number of the plurality of target virtual tasks has a passing performance.

6. The method of claim 1, wherein performance of each target virtual task of the plurality of target virtual tasks is associated with a score, wherein a score for the target virtual task indicates a level of performance of the associated target physical task by the therapy patient.

7. The method of claim 6, wherein determining that the therapy patient is able to perform the real-world activity successfully includes one or more of:
aggregating a score of each target virtual task of the plurality of target virtual tasks into an overall activity score, and determining that the overall activity score is above a passing threshold.

8. The method of claim 1, wherein the data indicative of performance of each target virtual task of the plurality of target virtual tasks includes one or more of:
a time taken to complete each target virtual task by the therapy patient; and
a number of fault indications occurring during performance of each target virtual task, wherein a fault indication occurring during performance of a target virtual task is configured to indicate a deviation from normal performance parameters by the therapy patient during performance of the target virtual task.

9. The method of claim 8, wherein generating the virtual environment configured to enable performance of the plurality of target virtual tasks includes:
configuring a virtual element of the virtual environment associated with a target virtual task with normal performance parameters;
configuring the virtual element associated with the target virtual task to detect the deviation from the normal performance parameters during performance of the target virtual task and to generate the fault indication in response to detecting the deviation from the normal performance parameters during performance of the target virtual task.

10. The method of claim 1, wherein generating the virtual environment includes mapping a surrounding associated with the therapy patient such that the plurality of target virtual tasks corresponding to the plurality of target physical tasks is performed within a physical environment associated with the therapy patient.

11. A system for simulating and assessing performance of real-world activities within a virtual environment by a therapy patient, comprising:
at least one processor; and
a memory operably coupled to the at least one processor and storing processor-readable code that, when executed by the at least one processor, is configured to perform operations including:
representing a real-world activity as a plurality of target physical tasks;
generating a plurality of target virtual tasks corresponding to the plurality of target physical tasks, wherein each of the plurality of target virtual tasks is associated with a target physical task of the plurality of target physical tasks such that a performance of a target virtual task of the plurality of target virtual tasks is indicative of a performance of a target physical task,
generating a virtual environment configured to enable performance of the plurality of target virtual tasks;
receiving data indicative of performance of each target virtual task of the plurality of target virtual tasks by the therapy patient; and
determining, based on the data indicative of the performance of each target virtual task of the plurality of target virtual tasks by the therapy patient, whether the therapy patient is able to perform the real-world activity successfully.

12. The system of claim 11, wherein performance of a first target virtual task of the plurality of target virtual tasks includes a wait period during which the therapy patient is to wait for completion of the first target virtual task without input by the therapy patient, and wherein generating the virtual environment configured to enable performance of the first target virtual task includes:
enabling performance of at least one target virtual task associated with another real-world activity different from the real-world activity during the wait period of the first target virtual task.

13. The system of claim 11, wherein generating the plurality of target virtual tasks corresponding to the plurality of target physical tasks includes one or more of:
generating the plurality of target virtual tasks as a sequential series of target virtual tasks, wherein a second target virtual task of the sequential series of target virtual tasks is prevented from being performed by the therapy patient until performance of a first target virtual task of the sequential series of target virtual tasks; and
generating the plurality of target virtual tasks as a non-sequential series of target virtual tasks, wherein each target virtual task of the non-sequential series of target virtual tasks is allowed to be performed by the therapy patient notwithstanding performance of other target virtual tasks of the non-sequential series of target virtual tasks.

14. The system of claim 11, wherein performance of each target virtual task of the plurality of target virtual tasks is pass or fail, wherein a passing performance of a target virtual task indicates successful completion of an associated target physical task by the therapy patient, and wherein a failing performance of the target virtual task indicates unsuccessful completion of the associated target physical task by the therapy patient.

15. The system of claim 14, wherein determining that the therapy patient is able to perform the real-world activity successfully includes one or more of:
determining that each target virtual task of the plurality of target virtual tasks has a passing performance; and
determining that a threshold number of the plurality of target virtual tasks has a passing performance.

16. The system of claim 11, wherein performance of each target virtual task of the plurality of target virtual tasks is associated with a score, wherein a score for the target virtual task indicates a level of performance of the associated target physical task by the therapy patient.

17. The system of claim 16, wherein determining that the therapy patient is able to perform the real-world activity successfully includes one or more of:
aggregating a score of each target virtual task of the plurality of target virtual tasks into an overall activity score, and determining that the overall activity score is above a passing threshold.

18. The system of claim 11, wherein the data indicative of performance of each target virtual task of the plurality of target virtual tasks includes one or more of:
a time taken to complete each target virtual task by the therapy patient; and
a number of fault indications occurring during performance of each target virtual task, wherein a fault indication occurring during performance of a target virtual task is configured to indicate a deviation from normal performance parameters by the therapy patient during performance of the target virtual task.

19. The system of claim 18, wherein generating the virtual environment configured to enable performance of the plurality of target virtual tasks includes:
configuring a virtual element of the virtual environment associated with a target virtual task with normal performance parameters;
configuring the virtual element associated with the target virtual task to detect the deviation from the normal performance parameters during performance of the target virtual task and to generate the fault indication in response to detecting the deviation from the normal performance parameters during performance of the target virtual task.

20. A computer-based tool for simulating and assessing performance of real-world activities within a virtual environment by a therapy patient, the computer-based tool including non-transitory computer readable media having stored thereon computer code which, when executed by a processor, causes a computing device to perform operations comprising:
representing a real-world activity as a plurality of target physical tasks;
generating a plurality of target virtual tasks corresponding to the plurality of target physical tasks, wherein each of the plurality of target virtual tasks is associated with a target physical task of the plurality of target physical tasks such that a performance of a target virtual task of the plurality of target virtual tasks is indicative of a performance of a target physical task,
generating a virtual environment configured to enable performance of the plurality of target virtual tasks;
receiving data indicative of performance of each target virtual task of the plurality of target virtual tasks by the therapy patient; and
determining, based on the data indicative of the performance of each target virtual task of the plurality of target virtual tasks by the therapy patient, whether the therapy patient is able to perform the real-world activity successfully.
